(19) United States
(12) Patent Application Publication
(10) Pub. No.: US 2004/0010178 A1
(43) Pub. Date: Jan. 15, 2004

(54) Magnetic head wear, hat, sweat band or eye glasses with thermo electric cooler or heater

(76) Inventor: Lynn A. Buckner, Chickamauga, GA (US)

Correspondence Address: LYNN A. BUCKNER, P.O. Box 609, Chickamauga, GA, 30707, US

(21) Appl. No.: 10/612,314

(22) Filed: Jul. 3, 2003

Related U.S. Application Data
(60) Provisional application No. 60/394,300, filed on Jul. 9, 2002.

(51) Int. Cl. A61N 2/00

(52) U.S. Cl. 600/9

(57) ABSTRACT

A magnetic head wear to place a magnetic field in close proximity a persons head, brain and blood flow. A magnetized hat, sweatband, magnetic eyeglasses, scarf, earmuffs, hearing aid or head ornament places a magnetic field in proximity to the brain which has significant blood volume & flow. Thus the magnetism improves the health of the body. A thermal cooler/heater may be added to a head gear to cool or warm the head thus improving body health.

### BACKGROUND OF THE INVENTION

Head bands, sweat band, caps or hats have not previously been equipped with magnetic means for the purpose of improving health.

### BRIEF SUMMARY OF THE INVENTION

The objective of the magnetic head wear is to place a magnetic field in close proximity a persons head, brain and blood flow. Wearing a magnetized hat, sweatband, magnetic eyeglasses, scarf, earmuffs, hearing aid or head ornament places a magnetic field in proximity to the brain which has significant blood volume & flow.

A magnetic field placed in close proximity to a human body for a period of time and or frequent intervals improves the health of the body. The above described devices, place a magnetic field in close proximity to the body of the person wearing the device and allows for frequent use of the device.

### BRIEF DISCRIPTION OF THE DRAWINGS

FIG. 1—Shows a HAT BAND 1 that is magnetized. The hat band 1 is a device constructed of a magnetic substance such as magnetic thread or a substance that has a magnet attached to it or a substance that has a magnet disposed within it. The magnet may be ground into small pieces and disposed within a substance to construct the hatband or larger magnets may be arranged in order to construct a hatband device. The hat or cap top 2 may also be magnetized. The hat or cap bill 3 may be magnetized.

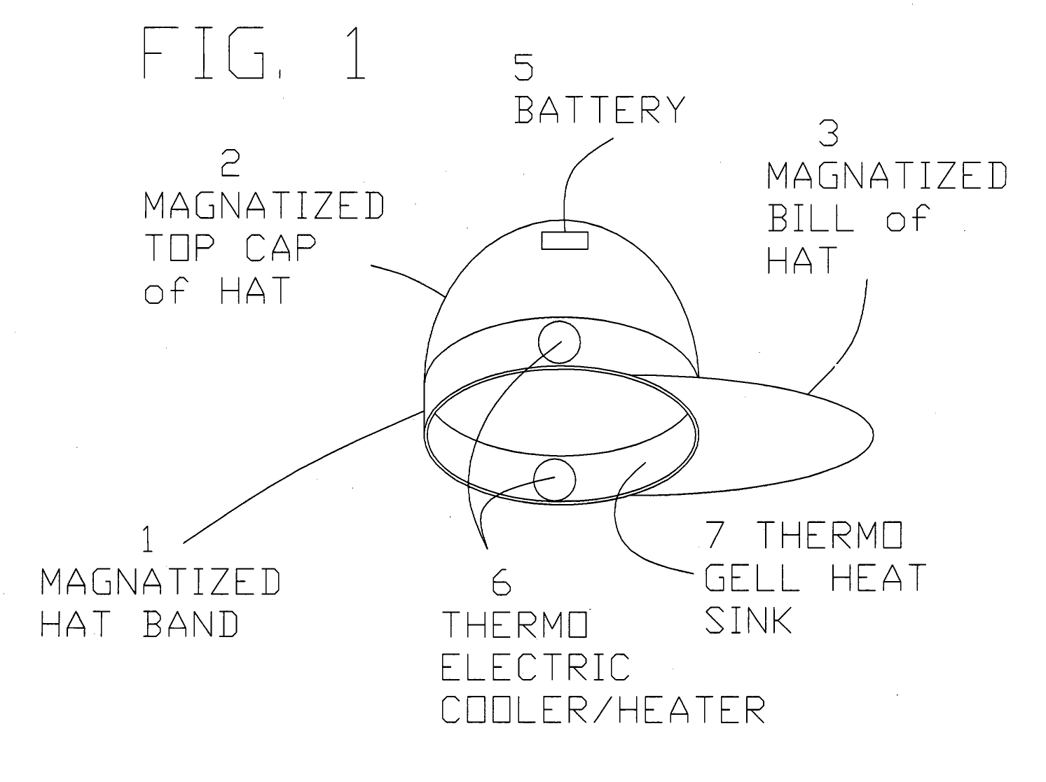

A thermoelectric cooler/heater 6 may be placed in proximity to the hat band. A battery 5 or other power source may be mounted in or on the hat to power the thermo electric heater/cooler.

FIG. 2—Show eye glasses 8 having magnetic means such as magnetic metal temples and or magnetic metal rems.

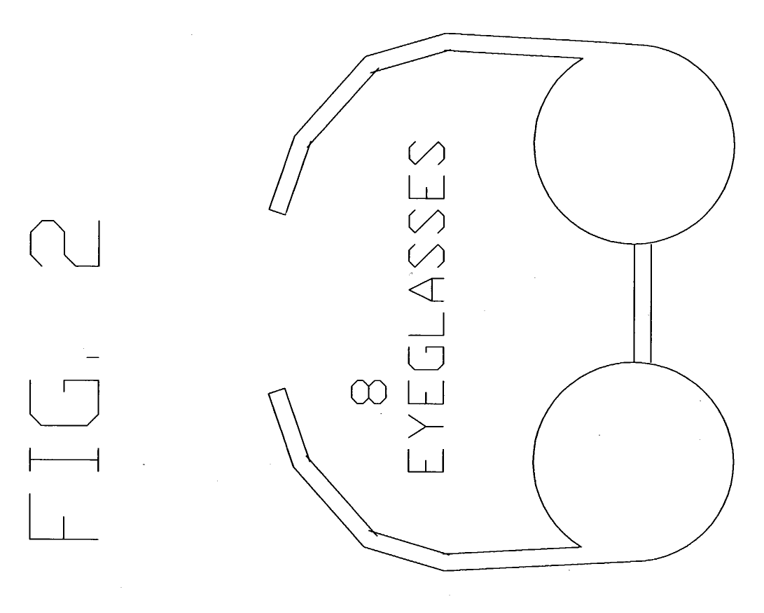

FIG. 3—Shows a magnetic sweat band 4 or head band.

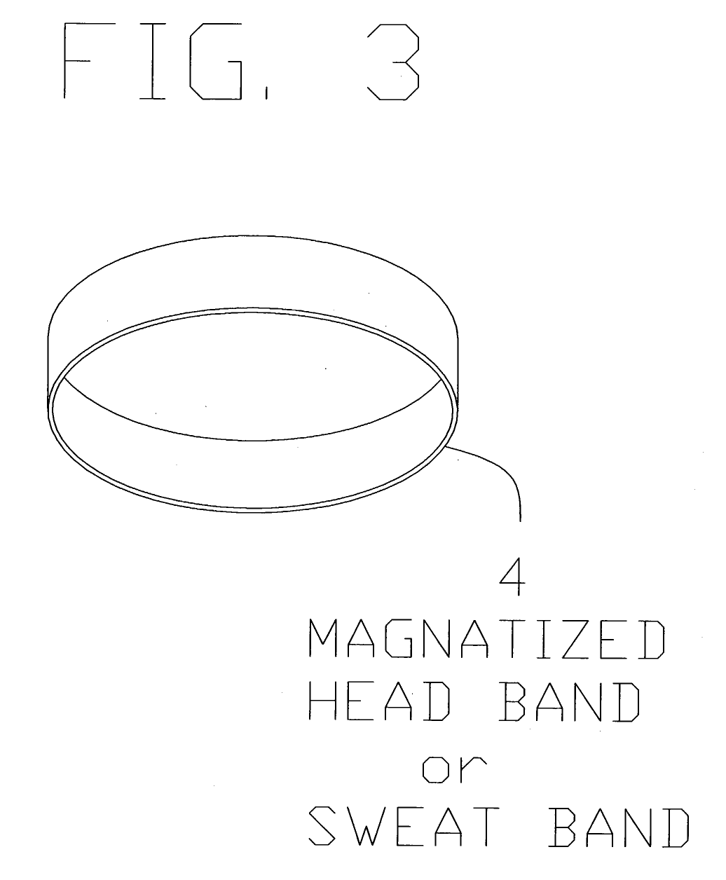

### DESCRIPTION OF A PREFERED IMBODIMENT

A hat or cap with a band 1 that is magnetized. The hat band 1 being a device constructed of a magnetic substance such as magnetic thread or a substance that has a magnet attached to it or a substance that has a magnet disposed within it. The magnet may be ground into small pieces and disposed within a substance to construct the hatband or larger magnets may be arranged in order to construct a hatband device. The hat or cap top 2 may also be magnetized. The hat or cap bill 3 may be magnetized.

A thermoelectric cooler/heater 6 may be placed in proximity to the hat band. A battery 5 or other power source may be mounted in or on the hat to power the thermo electric heater/cooler. A thermo conductor may also be added to the magnetic device to transfer energy either to or away from the person wearing the magnetic device. The heating device and/or cooling device may be chosen from a thermo electric cooler or a substance, which may be preheated or pre-cooled before wearing. The thermo electric cooler, air venturi, cooler or compressed gas cooler will cool or heat as required while being used by the person wearing the device.

A magnetic means may also be placed in proximity to the head by constructing magnetic properties in to eyeglasses, for example by making the temples or rims of a magnetic substance.

Hearing aids, head ornaments or ear muffs may also have magnetic properties attached or constructed as a part of them.

What is claimed

1. A magnetic means worn on or in proximity to the head.

2. Claim 1 wherein the head wear is a headband, or a hat or a cap or a sweat band or eyeglasses or earmuffs or hearing aid.

3. Claim 1 wherein the magnetic field created is beneficial to health.

4. Claim 1, where in a cooling device is added

5. claim 1, where in a heating device is added.

6. Claim 1, 2, or 3 wherein the device creating the magnetic field is a permanent magnet.

7. Claim 1, 2, 3, 4, or 5 wherein the device creating the magnetic field is an electro magnet.

8. Claim 1,2,3,4, 5, or 6 wherein a thermo electric cooler is added to the clothing garment.

9. Claim 1,2,3,4,5, or 6 wherein a thermo electric heater is added to the clothing garment.
